(19) United States
(12) Patent Application Publication
(10) Pub. No.: US 2019/0160084 A1
(43) Pub. Date: May 30, 2019

(54) METHODS FOR THE TREATMENT OF CANCER USING MEGLUMINE

(71) Applicants: Dynamis Pharmaceuticals, Inc., Jenkintown, PA (US); LANKANEU INSTITUTE FOR MEDICAL RESEARCH, Wynnewood, PA (US)

(72) Inventors: Annette TOBIA, Wyndmoor, PA (US); Alice MARCY, Doylestown, PA (US); Susan GILMOUR, Wynnewood, PA (US)

(21) Appl. No.: 16/320,725

(22) PCT Filed: Jul. 26, 2017
(86) PCT No.: PCT/US2017/043904

§ 371 Date: Jan. 25, 2019

Related U.S. Application Data
(60) Provisional application No. 62/366,815, filed on Jul. 26, 2016.

(51) Int. Cl. A61K 31/7008; A61K 9/00; A61K 9/70; A61P 35/00

(52) U.S. Cl. CPC A61K 31/7008; A61P 35/00; A61K 9/7023; A61K 9/0019

(57) ABSTRACT

Compositions and methods are disclosed for treating subjects with cancer, particularly skin cancer.

This application claims priority under 35 U.S.C. § 119(e) to U.S. Provisional Patent Application No. 62/366,815, filed Jul. 26, 2016. The foregoing application is incorporated by reference herein.

### FIELD OF THE INVENTION

This invention relates to the use of meglumine for the treatment of cancer, particularly skin cancer.

### BACKGROUND OF THE INVENTION

Several publications and patent documents are cited throughout the specification in order to describe the state of the art to which this invention pertains. Each of these citations is incorporated herein by reference as though set forth in full.

The rising incidence of skin cancer and its associated mortality, deformity and medical costs represent a major public health concern (Housman et al.). More than one million cases of nonmelanoma skin cancers are diagnosed each year within the United States alone (Rogers et al.). The rate of new diagnoses of melanoma rose 3.1% a year from 1992 to 2004 (Linos et al.). Although cutaneous malignant melanoma comprises only 4-11% of all skin cancers, it is responsible for more than 75% of skin cancer-related deaths (Netscher et al.). Patients with metastatic melanoma have median survival rates of only six to ten months. Thus, continued development and refinement of methods for early detection, prevention, and treatment of skin cancers are important goals of biomedical research.

Malignant skin tumors in humans include malignant melanoma, basal cell carcinoma (BCC) and squamous cell carcinoma (SCC) (Klein-Szanto; Dlugosz et al.). BCCs are the most common human cutaneous malignancy and they are typically slow-growing, locally invasive tumors that rarely metastasize. Although SCCs only represent about 20% of nonmelanoma skin cancers in humans, SCCs are generally more aggressive than the more common basal cell carcinomas (BCC) and can be lethal. Approximately 60% of SCCs arise from a preexisting benign actinic keratoses (Marks et al.). While most actinic keratoses do not progress to SCC, actinic keratoses represent SCC in situ at its earliest stages (Cockerell).

### SUMMARY OF THE INVENTION

In accordance with the instant invention, methods for inhibiting or treating cancer in a subject are provided. The method comprises administering meglumine (N-methylglucamine) or a pharmaceutically acceptable meglumine salt to the subject. In a particular embodiment, the cancer is a skin cancer. In a particular embodiment, the skin cancer is melanoma, squamous cell carcinoma, basal cell carcinoma, Bowen's disease, or actinic keratosis. The meglumine may be administered to the subject by any means. In a particular embodiment, the meglumine is administered orally. In a particular embodiment, is administered by injection (e.g., intraperitoneally or intravascularly). In a particular embodiment, the meglumine is administered topically (e.g., by transdermal patch). In a particular embodiment, the meglumine is administered in a dose sufficient to obtain a concentration of at least about 0.1 micromolar of meglumine in the blood of the subject (e.g., about 0.1 to about 100 micromolar). In a particular embodiment, the meglumine is administered from about 0.1 mg/kg to about 500 mg/kg per day based on the weight of the subject. The meglumine may be administered to a subject in the form of a composition that additionally comprises a pharmaceutically acceptable carrier.

### BRIEF DESCRIPTION OF THE DRAWINGS

FIG. 1 provides a graph of the pharmacokinetics of a single dose of meglumine hydrochloride in mice after oral (PO) administration (500 mg/kg in 0.25 ml) or intraperitoneal (IP) administration (100 mg/kg in 0.5 ml).

FIG. 2 provides a graph of the percentage of K6/ODC transgenic mice with skin tumors after receiving water (control) or meglumine.

FIG. 3 provides a graph of the average number of skin tumors (±SEM) in K6/ODC transgenic mice after receiving water (control) or meglumine.

FIG. 4 provides a graph of the average total skin tumor volume per mouse (±SEM) in K6/ODC transgenic mice after receiving water (control) or meglumine.

FIGS. 5A, 5B, 5C, 5D, 5E, and 5F provide graphs of the amount of IL-17, IL-8, MIP-1α (MIP-1a), MIP-1β (MIP-1b), IL-9 and IP-10, respectively, in the media of THP-1 cells treated with 25 ng of lipopolysaccharide (LPS) in the presence or absence of 40 or 80 mM meglumine hydrochloride.

### DETAILED DESCRIPTION OF THE INVENTION

### Initiation-Promotion Model of Skin Tumorigenesis

Whereas actinic keratoses are thought to be the benign precursor for human SCCs, papillomas have been identified as benign precursors for murine SCCs. Papillomas are benign epidermal tumors seen very frequently after chemical carcinogen exposure, especially in two-stage carcinogenesis protocols in mouse skin (Klein-Szanto et al.). Murine papillomas are cauliflower-like structures with a series of folds consisting of a central vascularized, connective tissue core covered by a proliferative, stratified squamous epithelium and an abundant, orthokeratotic horny layer. Some papillomas regress, but others can progress to malignancy. Squamous cell carcinomas (SCC) can be induced in animals using UV light, ionizing radiation, or chemical carcinogens. One of the most frequently used animal models for studying epithelial carcinogenesis is the initiation-promotion model of tumorigenesis in mouse skin. This model consists of initiation with a single application of the carcinogen, 7,12-dimethylbenz[a]anthracene (DMBA) followed by multiple topical treatments with a tumor promoting agent such as 12-O-tetradecanoylphorbol-13-acetate (TPA). Repeated TPA treatment clonally expands keratinocytes having a DMBA initiating c-Ha-ras mutation and leads to the growth of benign papillomas. Some of these papillomas eventually convert to malignant carcinomas.

### Polyamines and Tumorigenesis

Polyamines have long been known to be associated with cell proliferation in both normal and neoplastic tissues (Pegg 1986, 1988; Tabor et al.). Tightly regulated metabolism of polyamines is critical for cell survival and normal skin homeostasis, and these controls are dysregulated in tumorigenesis. Polyamines are biosynthesized from two amino acids, arginine and methionine. A key enzyme in polyamine biosynthesis, ornithine decarboxylase (ODC) is upregulated in the majority of tumors compared to normal tissue. Elevated levels of ODC and increased polyamines were initially suspected to play a causal role in tumor development largely due to the early induction of ODC by tumor promoters (O'Brien; Gilmour et al. 1986, 1987), and to studies using inhibitors of ODC (Bollag; Verma et al.; Takigawa et al.; Weeks et al.). For instance, α-difluoromethylornithine (DFMO), which is a specific and irreversible inhibitor of ODC enzyme activity, inhibits the development of skin tumors resulting from the DMBA-initiation/TPA promotion mouse skin tumorigenesis model when DFMO is given during the promotion phase (Takigawa et al.; Weeks et al.). Although cellular mechanisms exist to tightly control the expression of ODC in normal cells, the regulation of ODC is altered in tumor cells, yielding constitutively high levels of ODC expression (O'Brien; Gilmour et al. 1986, 1987; Pegg et al. 1998) and subsequent increased levels of polyamines (Koza et al.). ODC activity and polyamine levels are dramatically elevated in human squamous cell carcinomas compared to adjacent normal skin tissue (Scalabrino et al.; Hietala et al.). This can result from the upregulation of ODC expression by oncogenes such as c-myc (Bello-Fernandez et al.; Ben-Yosef et al.), v-src (Holtta et al.), v-raf (Shantz et al.), or an activated Ras or RhoA (Shantz et al.). However, although some oncogenes can increase ODC activity, ODC is also transiently induced in the skin by a variety of stimuli including mitogens, tumor promoters such as TPA, and hormones.

### K6/ODC Transgenic Mouse Model

Use of transgenic mouse models has demonstrated that polyamines play an essential role in the early promotional phase of skin tumorigenesis (Pegg et al. 2003; Gerner et al.). In order to mimic the constitutively elevated ODC enzyme activity and polyamine levels found in tumors, a variety of transgenic mice with keratin promoter-driven overexpression of key polyamine regulatory proteins have been used in the mouse skin chemical carcinogenesis model (Gilmour). Elevated ODC activity in K6/ODC transgenic mice has been constitutively targeted to either the outer root sheath of hair follicles in the skin using a keratin 6 promoter to yield increases in polyamine pools, especially putrescine levels (Megosh et al.; O'Brien et al.). These sustained high levels of putrescine lead to alopecia, the development of follicular dermal cysts, increased nail growth, and skin wrinkling. With regard to tumor development, the targeted expression of ODC to the skin also increases the susceptibility of these mice to skin tumor formation following a variety of initiating events including carcinogens (O'Brien et al.; Chen et al.), UV irradiation (Ahmad et al.), and oncogenes (Smith et al.; Lan et al. 2005; Tang et al.). K6/ODC transgenic mice develop skin tumors following a single, subthreshold dose of a carcinogen without the need of subsequent treatments with tumor promoting agents (i.e. TPA), as are needed in normal littermates or wild type mice. For instance, the treatment of newborn or adult K6/ODC transgenic mice with a single topical application of the carcinogen 7,12-dimethylbenz[a]anthracene (DMBA) yields papilloma formation six to eight weeks later without the use of tumor promoting agents (O'Brien et al.; Peralta Soler et al.). Likewise, single treatment with other carcinogens from different chemical classes also induces skin tumors in K6/ODC transgenic mice (Chen et al.). With particular relevance to human skin tumorigenesis, K6/ODC transgenic mice develop more papillomas following UVB irradiation compared to wildtype littermates (Ahmad et al.).

The formation of skin tumors in K6/ODC transgenic mice is dependent upon polyamine biosynthesis, especially putrescine, since treatment with inhibitors of ODC activity blocks the formation of skin tumors and causes the rapid regression of existing tumors (Peralta Soler et al.; Lan et al. 2000). Although the mechanism(s) by which polyamines promote skin tumorigenesis are not well understood, elevated levels of polyamines have been shown to stimulate epidermal proliferation, alter keratinocyte differentiation status, increase neovascularization, alter the immune response, and increase synthesis of extracellular matrix proteins in a manner similar to that seen in wound healing. For instance, increased expression of IL-1β and keratin 6, both markers of keratinocyte activation seen in wound healing, is also observed following de novo induction of epidermal ODC activity in adult, quiescent skin of ODCER transgenic mice, in which an inducible form of ODC is targeted to the epidermis (Lan et al. 2005). It is becoming increasingly apparent that elevated polyamine levels activate not only epidermal cells but also underlying stromal cells in the skin to promote the development and progression of skin tumors. Not surprisingly, the inhibition of polyamine biosynthesis has been shown to be a potent chemoprevention strategy for cancer. DFMO has been used in skin cancer chemoprevention trials that focus on patients with precancerous lesions such as actinic keratoses (Alberts et al.; Einspahr et al.) and sporadic colorectal adenomas (Meyskens et al.).

### Inflammation and Cancer

Cancer has a strong association with infectious agents, as shown by the increased incidence of cancers after human papilloma virus, hepatitis B and C and Kaposi's sarcoma human herpesvirus-8 infections. Similarly, inflammation can lead to an increase in organ cancer as seen in individuals with prostatitis, ulcerative colitis or pancreatitis. The inflammatory process results in recruitment of lymphoid cells, macrophages, and release of inflammatory cytokines that cause increased vascular permeability, attraction of phagocytic cells and cell death. Cytokines that mediate inflammation include IL-1, IL-2, IL-3, IL-4, IL-5, IL-6, IL-7, IL-8, IL-9, IL-10, IL-12, IL-13, IL-17, MIP-1 alpha, MIP-1 beta, IP-10, interferons and TNF. Anti-inflammatory agents, including nonsteroidal anti-inflammatory drugs (NSAIDS), corticosteroids and cyclooxygenase-2 inhibitors have shown anti-cancer properties (Rayburn et al.). NSAID users have a decreased occurrence of actinic keratosis, squamous cell carcinomas, and basal cell carcinomas (Butler et al.; Muranushi et al.; Reinau et al.).

In accordance with the instant invention, methods for inhibiting or treating cancer in a subject are provided. The method comprises administering meglumine (also known as, or (2R,3R,4R,5S)-6-methylaminohexane-1,2,3,4,5-pentol) or a pharmaceutically acceptable meglumine salt to the subject. In a particular embodiment, the cancer is a skin cancer (e.g., melanoma, squamous cell carcinoma, basal cell carcinoma, Bowen's disease, or actinic keratosis). The meglumine may be administered to the subject by any means (e.g., orally; by injection (e.g., intraperitoneally or intravascularly); topically (e.g., by transdermal patch)). In a particular embodiment, the meglumine is administered in a dose sufficient to obtain a concentration of at least about 0.1 micromolar of meglumine in the blood of the subject (e.g., about 0.1 to about 100 micromolar). In a particular embodiment, the meglumine is administered from about 0.1 mg/kg to about 500 mg/kg per day based on the weight of the subject. The meglumine may be administered to a subject in the form of a composition that additionally comprises a pharmaceutically acceptable carrier.

The instant invention includes a composition comprising meglumine or a salt thereof, wherein the composition is suitable for therapeutic administration to a subject. For example, the composition may further comprise a pharmaceutically acceptable carrier. The composition may further comprise an additional therapeutic agent (e.g., chemotherapeutic agent).

Meglumine may form salts with acids, and such salts are included in the present invention. In one embodiment, the salts are acceptable non-toxic salts. The term “salts” embraces addition salts of free acids useful within the methods of the invention. The term “acceptable salt” refers to salts that possess toxicity profiles within a range that affords utility in in vivo applications. Unacceptable salts may nonetheless possess properties such as high crystallinity, which have utility in the practice of the present invention, such as for example utility in process of synthesis, purification or formulation of compounds useful within the methods of the invention.

Suitable acceptable acid addition salts may be prepared from an inorganic acid or from an organic acid. Examples of inorganic acids include, without limitation: sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include, without limitation: formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

The compositions useful for the practice of the invention are in a form suitable for administration to a subject. Such compositions may comprise one or more acceptable carriers, one or more additional ingredients, or some combination of these. The meglumine may be present in the composition in the form of a physiologically acceptable salt, such as in combination with a physiologically acceptable cation or anion, as is well known in the art.

In an embodiment, the compositions useful for practicing the method of the invention may be administered to deliver a dose of between 1 ng/kg/day and 1000 mg/kg/day. In another embodiment, the compositions useful for practicing the invention may be administered to deliver a dose of between 0.1 mg/kg/day and 500 mg/kg/day.

The relative amounts of the active ingredient, the acceptable carrier, and any additional ingredients in a composition of the invention varies, depending upon the identity, size, and condition of the subject treated and further depending upon the route by which the composition is to be administered. By way of example, the composition may comprise between 0.01% and 100% (w/w) active ingredient.

Compositions useful in the methods of the invention may be suitably developed for inhalational, oral, rectal, vaginal, parenteral, topical, transdermal, pulmonary, intranasal, buccal, ophthalmic, intrathecal, parenteral, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the condition being treated, the type and age of the veterinary or human patient being treated, and the like.

The formulations of the compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multidose unit.

As used herein, a “unit dose” is a discrete amount of the composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (for example, about 1 to 4 or more times per day). When multiple daily doses are used, the unit dosage form may be the same or different for each dose.

Although the descriptions of compositions provided herein are principally directed to non-toxic compositions suitable for ethical administration to humans, it is understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and perform such modification with merely ordinary, if any, experimentation. Subjects to which administration of the compositions of the invention is contemplated include, but are not limited to, humans and other primates, mammals including commercially relevant mammals such as cattle, pigs, horses, sheep, cats, and dogs.

In one embodiment, the compositions are formulated using one or more acceptable non-toxic excipients or carriers. In one embodiment, the compositions comprise an effective amount of meglumine or a salt thereof and an acceptable carrier. Acceptable carriers, which are useful, include, but are not limited to, glycerol, water, saline, ethanol and other acceptable salt solutions such as phosphates and salts of organic acids. Examples of these and other acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Formulations may be employed in admixtures with conventional excipients, i.e., acceptable non-toxic organic or inorganic carrier substances suitable for oral, parenteral, nasal, intravenous, subcutaneous, enteral, or any other suitable mode of administration, known to the art. The preparations may be sterilized and if desired mixed with auxiliary agents, for example, lubricants, preservatives, stabilizers, wetting agents, emulsifiers, salts for influencing osmotic pressure buffers, coloring, flavoring and/or aromatic substances and the like. They may also be combined where desired with other active agents, for example, other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and acceptable polymeric or hydrophobic materials. Other “additional ingredients” that may be included in the compositions of the invention are known in the art and described, for example, in Genaro, ed., 1985, Remington's Pharmaceutical Sciences, Mack Publishing Co., Easton, Pa., which is incorporated herein by reference.

The composition of the invention may comprise a preservative, for example, from about 0.005% to 2.0% by total weight of the composition. The preservative is used to prevent spoilage in the case of exposure to contaminants in the environment. Examples of preservatives useful in accordance with the invention included, but are not limited to, those selected from the group consisting of benzyl alcohol, sorbic acid, parabens, imidurea and combinations thereof. A particular preservative is a combination of about 0.5% to 2.0% benzyl alcohol and 0.05% to 0.5% sorbic acid.

The composition may include an antioxidant and a chelating agent that inhibit the degradation of the compound. Antioxidants include, for example, BHT, BHA, alpha-tocopherol and ascorbic acid (e.g., in the range of about 0.01% to 0.3%, particularly BHT in the range of 0.03% to 0.1%, by weight by total weight of the composition. The chelating agent may be present in an amount of from 0.01% to 0.5% by weight by total weight of the composition. Chelating agents include, without limitation: edetate salts (for example, disodium edetate) and citric acid (e.g., in the weight range of about 0.01% to 0.20% or in the range of 0.02% to 0.10% by weight by total weight of the composition). The chelating agent is useful for chelating metal ions in the composition, which may be detrimental to the shelf life of the formulation. While BHT and disodium edetate are exemplified antioxidant and chelating agents, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (for example, polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one that comprises a carbon-containing liquid molecule and which exhibits a less polar character than water. Liquid solutions of the composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. For example, the therapeutic formulations may be administered to the subject either prior to or after diagnosis of any of the conditions contemplated herein, or to affect or improve any of the physiological functions contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal or human, may be carried out using known procedures, at dosages and for periods of time effective to treat the condition s contemplated herein in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the condition, age, sex, weight, condition, general health and prior medical history of the patient being treated, and like factors well-known in the art. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 500 mg/kg or about 0.1 and 500 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the condition being treated, and the type and age of the animal.

Actual dosage levels of the active ingredients in compositions of this invention may be varied so as to obtain an amount of the active ingredient that is effective to achieve the desired response for a particular patient, composition, and mode of administration, without being toxic to the patient.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of platelet hyperactivity in a patient.

In one embodiment, the compositions used for practicing the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from subject to subject depending on many factors including, but not limited to, age, condition to be treated, gender, overall health, and other factors. Thus, the invention should not be construed to be limited to any particular dosage regime and the precise dosage and composition to be administered to any patient will be determined by the attending physical taking all other factors about the patient into account.

The method of the invention involves administration of the therapeutic agent in the range, for example, of from about 1 μg to about 8,000 mg, about 20 μg to about 8,000 mg, about 40 μg to about 7,500 mg, about 80 μg to about 7,000 mg, about 100 μg to about 6,500 mg, about 200 μg to about 6,000 mg, about 400 μg to about 5,000 mg, about 800 μg to about 4,000 mg, about 1 mg to about 3,500 mg, about 2 mg to about 3,000 mg, about 5 mg to about 2,000 mg, about 10 mg to about 1,000 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In one embodiment, the present invention is directed to a packaged composition comprising a container holding a therapeutically effective amount of a compound of the invention, alone or in combination with a second therapeutic agent; and instructions for using the compound to treat, prevent, or reduce the conditions contemplated herein in a patient.

Routes of administration of any of the compositions of the invention include, without limitation: inhalational, oral, nasal, rectal, parenteral, sublingual, transdermal, transmucosal (for example, sublingual, lingual, (trans)buccal, (trans)urethral, vaginal (for example, trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, without limitation: tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gel caps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic excipients suitable for the manufacture of tablets. Such excipients include, for example, an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for a non-toxic and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with non-toxic acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (for example, OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with acceptable non-toxic additives such as suspending agents (for example, sorbitol syrup, methyl cellulose or hydrogenated edible fats); emulsifying agent (for example, lecithin or acacia); non-aqueous vehicles (for example, almond oil, oily esters or ethyl alcohol); and preservatives (for example, methyl or propyl para-hydroxy benzoates or sorbic acid). Liquid formulations of a non-toxic composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, an acceptable non-toxic carrier, and at least sufficient liquid to moisten the mixture. Acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active compound by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

As used herein, “parenteral administration” of a composition includes any route of administration characterized by physical breaching of a tissue of a subject and administration of the composition through the breach in the tissue. Parenteral administration thus includes, but is not limited to, administration of a composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a composition suitable for parenteral administration comprise the active ingredient combined with an acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (for example, sterile pyrogen-free water) prior to parenteral administration of the reconstituted composition.

The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or diglycerides. Other parentally-administrable formulations useful include those which comprise the active ingredient in microcrystalline form, in a liposomal preparation, or as a component of a biodegradable polymer system. Compositions for sustained release or implantation may comprise acceptable non-toxic polymeric or hydrophobic materials such as an emulsion, an ion exchange resin, a sparingly soluble polymer, or a sparingly soluble salt.

An obstacle for topical administration of compositions is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of compounds across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, polar lipids, or N-methyl-2-pyrrolidone.

One acceptable vehicle for topical delivery of some of the compositions of the invention may contain liposomes. The composition of the liposomes and their use are known in the art (for example, U.S. Pat. No. 6,323,219).

In alternative embodiments, the topically active composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active composition should be applied in an amount effective to affect desired changes. As used herein “amount effective” shall mean an amount sufficient to cover the region of skin surface where a change is desired. An active compound may be present in the amount of, for example, from about 0.0001% to about 15% by weight volume of the composition; from about 0.0005% to about 5% of the composition; or from about 0.001% to about 1% of the composition. Such compounds may be synthetically- or naturally derived.

A composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

A composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating acceptable excipient which is solid at ordinary room temperature (i.e., about 20° C.) and which is liquid at the rectal temperature of the subject (i.e., about 37° C. in a healthy human). Suitable non-toxic acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a non-toxic acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

Controlled- or sustained-release formulations of a non-toxic composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, may be readily selected for use with the compositions of the invention. Thus, single unit dosage forms suitable for oral administration, such as tablets, capsules, gel caps, and caplets, which are adapted for controlled-release are encompassed by the present invention.

Most controlled-release products have a common goal of improving therapy over that achieved by their non-controlled counterparts. Ideally, the use of an optimally designed controlled-release preparation in treatment is characterized by a minimum of substance being employed to cure or control the condition in a minimum amount of time. Advantages of controlled-release formulations include extended activity of the compound, reduced dosage frequency, and increased subject compliance. In addition, controlled-release formulations can be used to affect the time of onset of action or other characteristics, such as blood level of the compound, and thus can affect the occurrence of side effects.

Most controlled-release formulations are designed to initially release an amount of compound that promptly produces the desired therapeutic effect, and gradually and continually release of other amounts of compound to maintain this level of therapeutic effect over an extended period of time. In order to maintain this constant level of compound in the body, the compound must be released from the dosage form at a rate that will replace the amount of compound being metabolized and excreted from the body.

Controlled-release of an active ingredient can be stimulated by various inducers, for example, pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a formulation that provides for gradual release of a compound over an extended period of time, and that may, although not necessarily, result in substantially constant blood levels of a compound over an extended time period. The period of time may be as long as a month or more and should be a release which is longer that the same amount of agent administered in bolus form. For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation. In a particular embodiment of the invention, the compounds of the invention are administered to a patient, alone or in combination with another agent, using a sustained release formulation.

The term delayed release is used herein in its conventional sense to refer to a formulation that provides for an initial release of the compound after some delay following compound administration and that mat, although not necessarily, includes a delay of from about 10 minutes up to about 12 hours. The term pulsatile release is used herein in its conventional sense to refer to a formulation that provides release of the compound in such a way as to produce pulsed plasma profiles of the compound after compound administration. The term immediate release is used in its conventional sense to refer to a formulation that provides for release of the compound immediately after compound administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after compound administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after compound administration.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The singular forms “a,” “an,” and “the” include plural referents unless the context clearly dictates otherwise.

As used herein when referring to a measurable value such as an amount, a temporal duration, and the like, the term “about” is meant to encompass variations of ±20% or ±10%, particularly ±5%, ±1%, or ±0.1% from the specified value, as such variations are appropriate to perform the disclosed methods.

As used herein, a “subject” or “individual” or “patient” may be a human or non-human mammal. Non-human mammals include, for example, livestock and pets, such as ovine, bovine, porcine, canine, feline and murine mammals. In a particular embodiment, the subject is human.

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to treat cancer in a subject.

As used herein, the term “acceptable” refers to a material, such as a carrier or diluent, which does not abrogate the biological activity or properties of the compound or composition, and is relatively non-toxic, i.e., the material may be administered to an individual without causing undesirable biological effects or interacting in a deleterious manner with any of the components of the composition in which it is contained.

As used herein, the language “acceptable salt” refers to a salt of the administered compounds prepared from acceptable non-toxic acids, including, without limitation, inorganic acids, organic acids, solvates, hydrates, or clathrates thereof.

As used herein, the term “composition” refers to a mixture of at least one compound useful within the invention with other chemical components, such as carriers, stabilizers, diluents, dispersing agents, suspending agents, thickening agents, and/or excipients. The composition facilitates administration of the compound to an organism. Multiple techniques of administering a compound exist in the art including, but not limited to: intravenous, oral, aerosol, parenteral, ophthalmic, pulmonary and topical administration. In one embodiment, the composition is non-toxic.

The language “acceptable carrier” includes an acceptable salt, acceptable material, composition or carrier, such as a liquid or solid filler, diluent, excipient, solvent or encapsulating material, involved in carrying or transporting a compound(s) of the present invention within or to the subject such that it may perform its intended function. The acceptable carrier is preferably non-toxic. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in non-toxic formulations, or any combination thereof. As used herein, “acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) or purpose of the claimed invention. All methods described herein that embody the present invention can, in alternate embodiments, be more specifically defined by any of the transitional terms “comprising,” “consisting essentially of,” and “consisting of.”

The following examples are provided to illustrate certain embodiments of the invention. They are not intended to limit the invention in any way.

### Example I

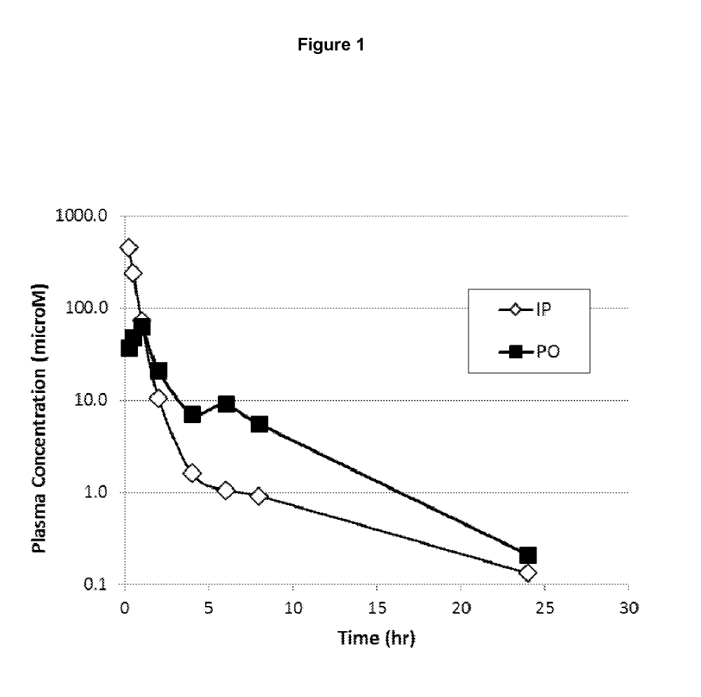

The pharmacokinetics of a single dose of meglumine hydrochloride was measured in male, HLA Swiss mice weighing 20-30 grams (FIG. 1). Animals (24 in each group) were dosed either by oral gavage (500 mg/kg in 0.25 ml) or intraperitoneally (100 mg/kg in 0.5 ml). A terminal brachial bleed of three mice was taken after 15 minutes, 30 minutes, 1 hour, 2 hour, 4 hour, 6 hour, 8 hour, and 24 hour of dosing. The pooled blood was collected into potassium EDTA coated tubes. Approximately 1 ml of blood was collected to obtain a 500 microliter sample of plasma. The tubes were spun for approximately 10 minutes at 8000 rpm, and plasma was drawn off into plastic tubes for storage at −80° C. prior to analysis.

Meglumine concentrations in plasma were determined using liquid chromatography-tandem mass spectrometry (LC-MS/MS). Plasma samples (25 microliters) were combined with 25 microliters of acetonitrile:water (1:1), and 25 microliters of 4 microgram/ml glucosamine as an internal standard. Samples were combined with 200 microliters of methanol:acetonitrile (1:1), vortexed, and centrifuged at 3000 rpm for 10 minutes. The supernatant (50 microliters) was transferred to a clean plate containing 200 microliters of mobile phase B (0.2% AA, 0.05% TFA in acetonitrile). The LC used an Atlantis HILIC Silica, 50×3, 5 μm column with a flow rate of 0.8 ml/minute, and a gradient as described below in Table 1. Mass spectrometry parameters are provided in Table 2.

TABLE 1
Liquid chromatography parameters.

| LC gradient: | | | |
|---|---|---|---|
| Time | | HPLC System: | Shimadzu Prominence |
| (min) | % B | Column: | Atlantis HILIC Silica, 50 × 3, 5 μm |
| 0 | 92 | Flow rate: | 0.8 mL/minute |
| 0.2 | 92 | Mobile phase A: | 0.2% AA 0.05% TFA in water |
| 1 | 40 | Mobile Phase B: | 0.2% AA 0.05% TFA in Acetonitrile |
| 1.5 | 40 | Injector Wash: | Methanol:water 1:1 |
| 1.55 | 92 | Injection Vol: | 10 mL |
| 3.0 | Stop | | |

TABLE 2
MS/MS Parameters. Sciex API4000. ESI positive mode.

| | Parent | Product | Scan | | | | |
|---|---|---|---|---|---|---|---|
| Compound | m/z | m/z | (ms) | DP | CE | CXP | RT |
| meglumine | 196.2 | 44.1 | 100 | 50 | 48 | 12 | 1.7 min |
| Glucosamine | 180.2 | 72.1 | 100 | 38 | 25 | 12 | 1.7 min |

Other Detector Parameters:

| | | | | | | | |
|---|---|---|---|---|---|---|---|
| Ion Source | 500° C. | GS1: 50 | GS2: 50 | IS: 4500 | NC: | CAD: 6 | EP: 10 |
| Tem: | | | | | NA | | |

### Example 2

K6/ODC transgenic mice are highly susceptible to developing skin tumors following a single treatment with a low subthreshold dose of the carcinogen DMBA. The effect of oral administration of meglumine in the drinking water was tested on the formation and growth of skin tumors in DMBA-initiated K6/ODC transgenic mice and their normal littermates. Four day old K6/ODC transgenic mice and their normal littermates were initiated with a single topical application of 300 nmol DMBA in 50 μl acetone. At birth the dam was given 0.5% DFMO in her drinking water to suppress ODC activity in K6/ODC transgenic pups until weaned at 3 weeks of age when DFMO administration was stopped. DFMO has been shown to transfer to pups via the milk, and administration of this dose of DFMO has no adverse effects on the development of the mice.

Upon weaning, K6/ODC transgenic mice and their normal littermates were divided into two treatment groups where they received ad lib either tap water or water with 37.5 mM meglumine (Sigma Chemical Co.) in their drinking water. Meglumine-containing water was changed twice a week. Addition of meglumine to the drinking water did not affect the consumption of water nor did it have any adverse effects (i.e. weight loss, lethargy, ruffed fur, diarrhea, or general malaise) on the mice. Mice were monitored for tumor development. Tumor growth was assessed morphometrically using calipers, and tumor volumes were calculated according to the formula V (mm³)=π/6×A×B² (A is larger diameter, B is the smaller diameter) (Buzzai et al.).

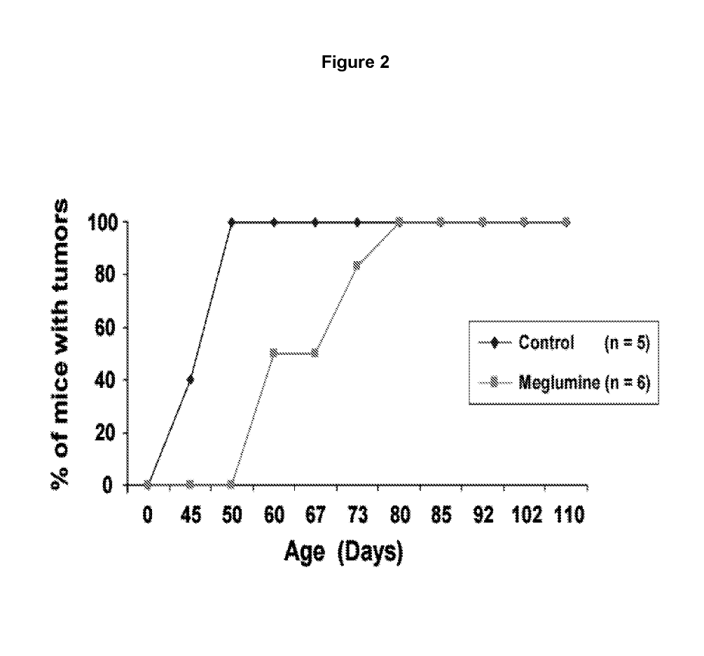

As previously reported (O'Brien et al.; Chen et al.), without repeated treatment with a tumor promoting agent such as TPA, no normal littermates [n=10] (with or without meglumine-supplemented water) developed skin tumors following initiation with this single, subthreshold dose of DMBA. Skin tumors first appeared in K6/ODC transgenic mice given control tap water at 45 days of age, and all (5/5) control K6/ODC transgenic mice had skin tumors by 50 days of age (FIG. 2). In contrast, no meglumine-treated K6/ODC transgenic mice developed skin tumors until 60 days of age, and not until 73 days of age did all (6/6) meglumine-treated mice have skin tumors (FIG. 2).

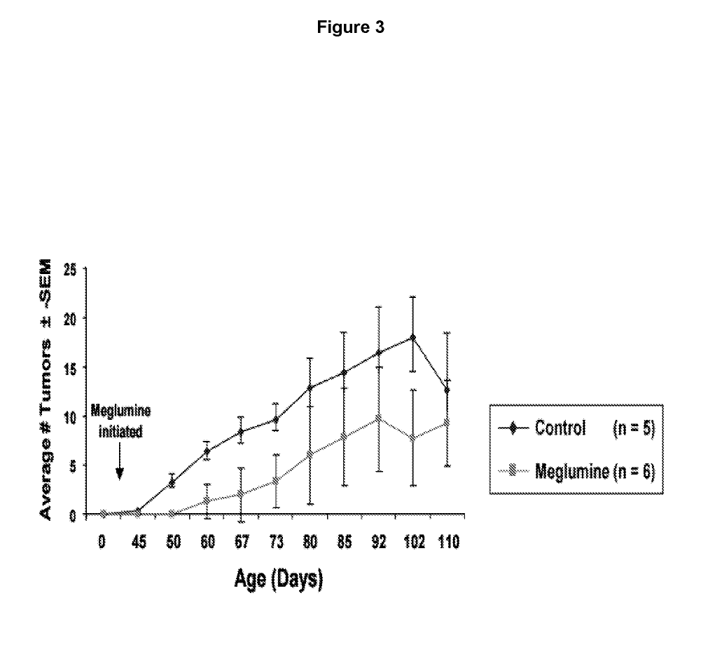

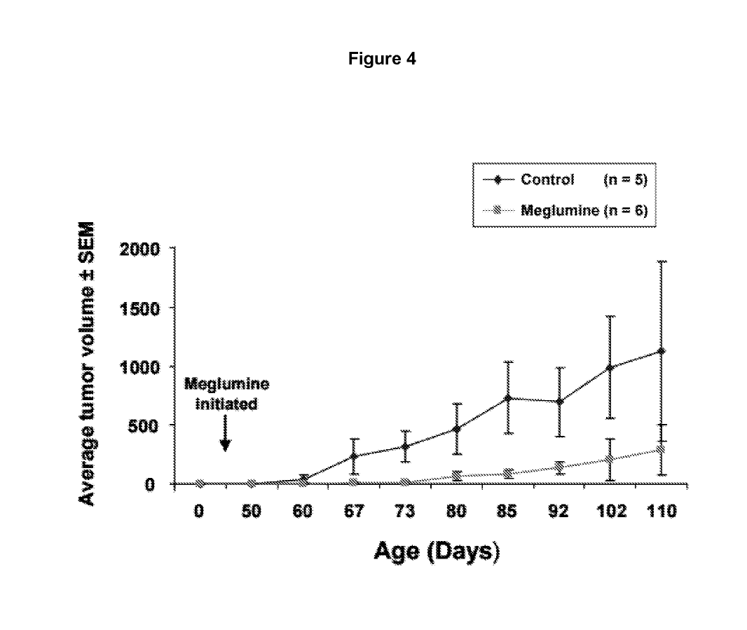

Mice were monitored for tumor number and tumor growth for 4 months. K6/ODC transgenic mice administered meglumine in the drinking water demonstrated fewer numbers of skin tumors at any given age compared to that in K6/ODC transgenic mice given control tap water (FIG. 3). In addition, tumor growth was slowed in meglumine-treated mice with a lower cumulative tumor burden (measured by average total tumor volume per mouse) compared to that in K6/ODC mice administered control water (FIG. 4).

These data show a protective effect of oral administration of meglumine to inhibit the incidence of DMBA-induced tumors, the number of skin tumors, and tumor growth in a transgenic mouse model that is highly susceptible to developing skin tumors. It is important to note that K6/ODC transgenic mice only develop skin tumors because the ODC transgene is directed to the skin. These tumors are polyamine-dependent since specific inhibition of the ODC transgene blocks skin tumor development in K6/ODC mice. However, all epithelial tumors have constitutively higher levels of polyamines, thus indicating that meglumine will have a protective effect in other types of epithelial tumors as well.

### Example 3

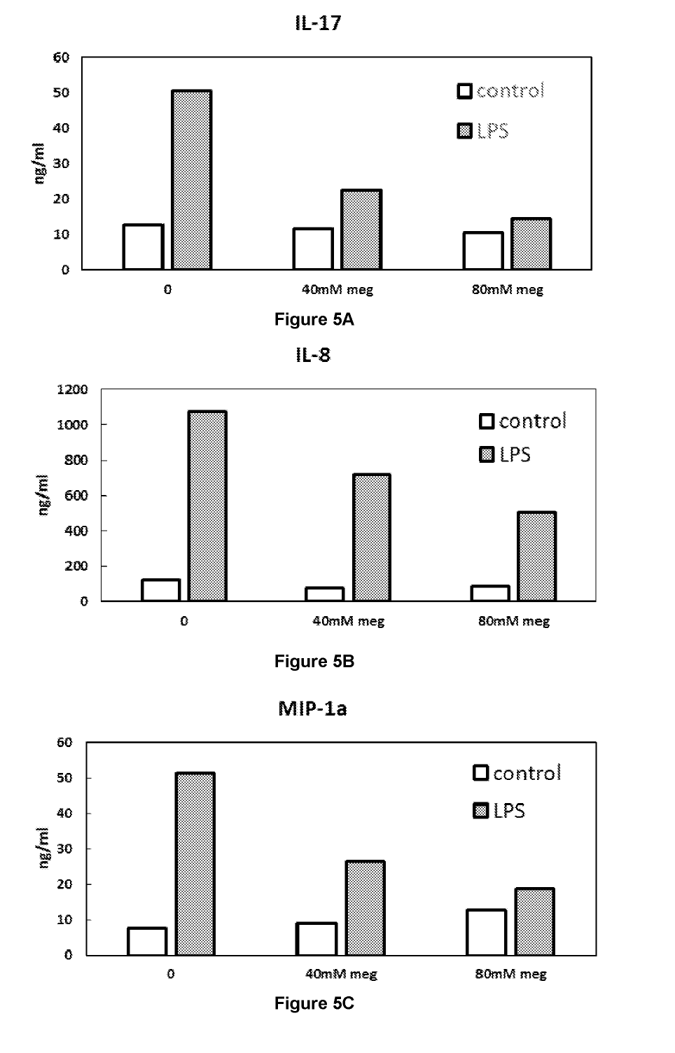

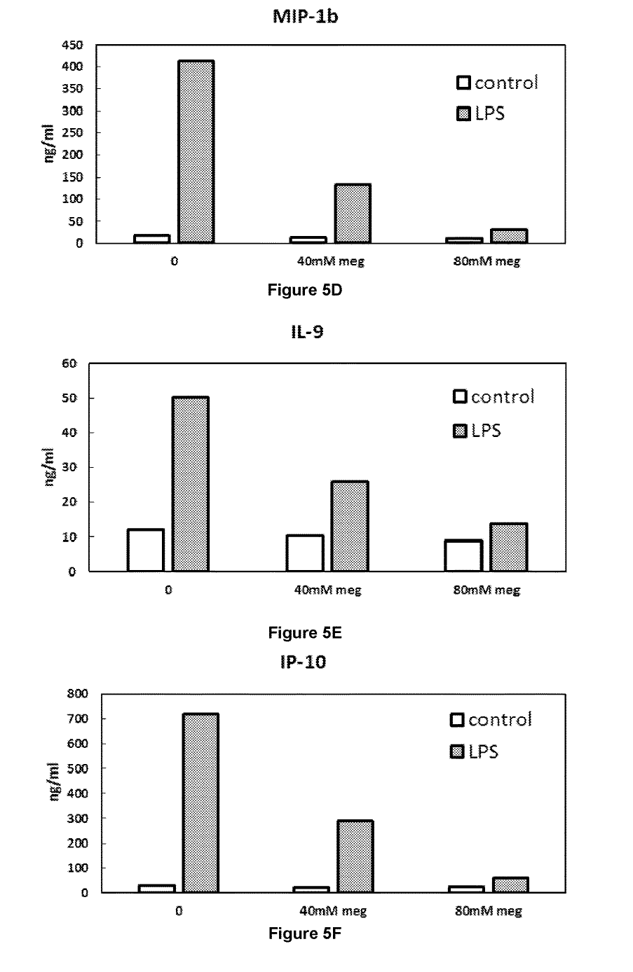

THP-1 cells (human acute monocytic leukemia) were grown in a 24-well polystyrene plate in 1 ml of RPMI media with 10% fetal bovine serum. Cells were treated with 25 ng of lipopolysaccharide (LPS) (Sigma) in the presence or absence of 40 or 80 mM meglumine hydrochloride. After 24 hours, the media was centrifuged to remove the cells and analyzed for cytokine content using a Bio-Plex® immunoassay (Bio-Rad). The LPS induced levels of IL-17, IL-8, MIP-1 alpha, MIP-1 beta, IL-9 and IP-10 were decreased in the presence of meglumine (FIG. 5).

### REFERENCES CITED

Ahmad et al., “A definitive role of ornithine decarboxylase in photocarcinogenesis.” Am. J. Pathol., 159:885-892, 2001.
Alberts et al. “Chemoprevention of human actinic keratoses by topical 2-(difluoromethyl)-dl-ornithine.” Cancer Epidemiol. Biomarkers Prev., 9:1281-1286, 2000.
Bello-Fernandez et al., “The ornithine decarboxylase gene is a transcriptional target of c-Myc.” Proc Natl Acad Sci USA 90:7804-7808, 1993.
Ben-Yosef et al., “Involvement of Myc targets in c-myc and N-myc induced human tumors.” Oncogene 17:165-171, 1998.
Bollag, W. “Prophylaxis of chemically induced benign and malignant epithelial tumors by vitamin A acid (retinoic acid).” Eur. J. Cancer 8:689-693, 1972.
Butler et al., “Nonsteroidal anti-inflammatory drugs and the risk of actinic keratoses and squamous cell cancers of the skin.” J. Am. Acad. Derm., 53:966-972, 2005.
Buzzai et al., “Systemic treatment with the antidiabetic drug metformin selectively impairs p53-deficient tumor cell growth.” Cancer Res., 67:6745-6752, 2007.
Chen et al., “K6/ODC transgenic mice as a sensitive model for carcinogen identification.” Toxicol. Lett., 116:27-35, 2000.
Cockerell, C. J., “Histopathology of incipient intraepidermal squamous cell carcinoma (“actinic keratosis”).” J. Am. Acad. Dermatol., 42:11-17, 2000.
Dlugosz et al., “Progress in cutaneous cancer research.” J. Investig. Dermatol. Symp. Proc., 7:17-26, 2002.
Einspahr et al., “Modulation of biologic endpoints by topical difluoromethylornithine (DFMO), in subjects at high risk for nonmelanoma skin cancer.” Clin. Cancer Res., 8:149-155, 2002.
Gerner et al., “Polyamines and cancer: old molecules, new understanding.” Nat. Rev. Cancer 4:781-792, 2004.
Gilmour, S. K. “Polyamines and nonmelanoma skin cancer.” Toxicol. Appl. Pharmacol., 224:249-256, 2007.
Gilmour et al., “Heterogeneity of ornithine decarboxylase expression in 12-O-tetradecanoylphorbol-13-acetate-treated mouse skin and in epidermal tumors.” Carcinogenesis 7:943-947, 1986.
Gilmour et al. “Regulation of ornithine decarboxylase gene expression in mouse epidermis and epidermal tumors during two-stage tumorigenesis.” Cancer Res., 47:1221-1225, 1987.
Hietala et al. “Activation of human squamous cell carcinoma ornithine decarboxylase activity by guanosine triphosphate.” Cancer Res., 48:1252-1257, 1988.
Holtta et al., “Polyamines are essential for cell transformation by pp60v-src delineation of molecular events relevant for the transformed phenotype.” J. Cell Biol., 122:903-914, 1993.
Housman et al., “Skin cancer is among the most costly of all cancers to treat for the Medicare population.” J. Am. Acad. Dermatol., 48:425-429, 2003.
Klein-Szanto, A. J. “Pathology of human and experimental skin tumors.” Carcinog Compr. Surv., 11:19-53, 1989.
Koza et al., “Constitutively elevated levels of ornithine and polyamines in mouse epidermal papillomas.” Carcinogenesis 12:1619-1625, 1991.
Lan et al., “Suprabasal induction of ornithine decarboxylase in adult mouse skin is sufficient to activate keratinocytes.” J. Invest. Dermatol., 124:602-614, 2005.
Lan et al., “Inhibition of ornithine decarboxylase (ODC) decreases tumor vascularization and reverses spontaneous tumors in ODC/Ras transgenic mice.” Cancer Res., 60:5696-5703, 2000.
Linos et al., “Increasing burden of melanoma in the United States.” J. Invest. Dermatol., 129: 1666-1674, 2009.
Marks et al., “Malignant transformation of solar keratoses to squamous cell carcinoma.” Lancet 1:795-797, 1988.
Megosh et al. “Increased frequency of spontaneous skin tumors in transgenic mice which overexpress ornithine decarboxylase.” Cancer Res., 55:4205-4209, 1995.
Meyskens et al., “Difluoromethylornithine plus sulindac for the prevention of sporadic colorectal adenomas: a randomized placebo-controlled, double-blind trial.” Cancer Prev. Res., 1:32-38, 2008.
Muranushi et al., “Aspirin and nonsteroidal anti-inflammatory drugs can prevent cutaneous squamous cell carcinoma: a systematic review and meta-analysis.” J. Invest. Derm., 135:975-983, 2015.
Netscher et al., “Cutaneous malignancies: melanoma and nonmelanoma types.” Plast. Reconstr. Surg., 127: 37e-56e, 2011.
O'Brien, T. G. “The induction of ornithine decarboxylase as an early, possibly obligatory event in mouse skin carcinogenesis.” Cancer Res., 36:2644-2653, 1976.
O'Brien et al., “Ornithine decarboxylase overexpression is a sufficient condition for tumor promotion in mouse skin.” Cancer Res., 57:2630-2637, 1997.
Pegg, A. E. “Recent advances in the biochemistry of polyamines in eukaryotes.” Biochem. J., 234:249-262, 1986.
Pegg, A. E. “Polyamine metabolism and its importance in neoplastic growth as a target for chemotherapy.” Cancer Res., 48:759-774, 1988.
Pegg et al., “Transgenic mouse models for studies of the role of polyamines in normal, hypertrophic and neoplastic growth.” Biochem. Soc. Trans., 31: 356-360, 2003.
Pegg et al., “S-adenosylmethionine decarboxylase: structure, function and regulation by polyamines.” Biochem. Soc. Trans., 26:580-6, 1998.
Peralta Soler et al. “Polyamines regulate expression of the neoplastic phenotype in mouse skin.” Cancer Res., 58:1654-1659, 1998.
Rayburn et al., “Anti-inflammatory agents for Cancer Therapy.” Mol. Cel. Pharmacol., 1:29-43, 2009.
Reinau et al., “Nonsteroidal anti-inflammatory drugs and the risk of non-melanoma skin cancer.” Int. J. Cancer, 137:144-153, 2015.
Rogers et al., “Incidence estimate of nonmelanoma skin cancer in the United States, 2006.” Arch. Dermatol. 146: 283-287, 2010.
Scalabrino et al. “Levels of activity of the polyamine biosynthetic decarboxylases as indicators of degree of malignancy of human cutaneous epitheliomas.” J. Invest. Dermatol., 74:122-124, 1980.
Shantz et al., “Ornithine decarboxylase induction in transformation by H-Ras and RhoA.” Cancer Res., 58: 2748-2753, 1998.
Smith et al., “Co-operation between follicular ornithine decarboxylase and v-Ha-ras induces spontaneous papillomas and malignant conversion in transgenic skin.” Carcinogenesis 19:1409-1415, 1998.
Tabor et al. “Polyamines.” Ann. Rev. Biochem., 53:749-790, 1984.
Takigawa et al. “Inhibition of mouse skin tumor promotion and of promoter-stimulated epidermal polyamine biosynthesis by alphadifluoromethylornithine.” Cancer Res., 43:3732-3738, 1983.
Tang et al. “Ornithine decarboxylase is a target for chemoprevention of basal and squamous cell carcinomas in Ptchl+/− mice.” J. Clin. Invest., 113:867-875, 2004.
Verma et al., “Inhibition by prostaglandin synthesis inhibitors of the induction of epidermal ornithine decarboxylase activity, the accumulation of prostaglandins and tumor promotion caused by 12-O-tetradecanoylphorbol-13-acetate.” Cancer Res., 40:308-315, 1980.
Weeks et al., “Alpha-Difluoromethylornithine, an irreversible inhibitor of ornithine decarboxylase, inhibits tumor promoter-induced polyamine accumulation and carcinogenesis in mouse skin.” Proc Natl Acad Sci USA, 79:6028-6032, 1982.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method for treating cancer in a subject in need thereof, said method comprising administering to the subject a therapeutically effective amount of meglumine or a pharmaceutically acceptable salt thereof.

2. The method of claim 1, wherein the cancer is a skin cancer.

3. The method of claim 2, wherein the skin cancer is melanoma, squamous cell carcinoma, basal cell carcinoma, Bowen's disease, or actinic keratosis.

4. The method of claim 1, wherein said meglumine or a pharmaceutically acceptable salt thereof is administered in the form of a composition additionally comprising a pharmaceutically acceptable carrier.

5. The method of claim 1, wherein said meglumine or a pharmaceutically acceptable salt thereof is administered orally.

6. The method of claim 1, wherein said meglumine or a pharmaceutically acceptable salt thereof is administered by a transdermal patch.

7. The method of claim 1, wherein said meglumine or a pharmaceutically acceptable salt thereof is administered intravenously.

8. The method of claim 1, wherein the dose administered to the subject is from 0.1 mg/kg to 500 mg/kg per day based on the weight of said subject.

9. The method of claim 1, wherein said meglumine or a pharmaceutically acceptable salt thereof is administered in a dose sufficient to obtain a meglumine concentration in the blood of said subject of about 0.1 to about 100 micromolar.

10. The method of claim 1, wherein said subject is a human.
